(19) United States
(12) Patent Application Publication
(10) Pub. No.: US 2025/0177959 A1
(43) Pub. Date: Jun. 5, 2025

(54) PARTICULATE CARBON MATERIAL PROVIDED WITH ELEMENTAL SILVER AND ELEMENTAL RUTHENIUM

(71) Applicant: HERAEUS PRECIOUS METALS GMBH & CO. KG, Hanau (DE)

(72) Inventors: Milena KOVJENIC, Hanau (DE); Ana-Lucia MEZA-CHINCHA, Hanau (DE); Marie-Lena HARWARDT, Hanau (DE); Michael GOCK, Hanau (DE); Matthias HÖRTEIS, Hanau (DE)

(21) Appl. No.: 18/840,977

(22) PCT Filed: Sep. 8, 2022
(86) PCT No.: PCT/EP2022/074937

§ 371 Date: Aug. 23, 2024

(30) Foreign Application Priority Data
Feb. 28, 2022 (EP) 22159080.5

(51) Int. Cl. B01J 23/50; A01N 25/12; A01N 59/16; A01P 1/00; B01J 21/18; B01J 35/30; B01J 35/40; B01J 35/61; B01J 35/63; B01J 37/06; B01J 37/08; B01J 37/16; C01B 32/372; C02F 1/50; C02F 1/72

(52) U.S. Cl. CPC B01J 23/50; A01N 25/12; A01N 59/16; A01P 1/00; B01J 21/18; B01J 35/394; B01J 35/40; B01J 35/615; B01J 35/617; B01J 35/618; B01J 35/635; B01J 35/638; B01J 37/06; B01J 37/08; B01J 37/16; C01B 32/372; C02F 1/50; C02F 1/725; C01P 2004/60; C01P 2006/12; C01P 2006/14; C02F 2303/04; C02F 2305/023

(57) ABSTRACT

A particulate carbon material that is provided with elemental silver and elemental ruthenium and has an average particle size (d50) in the range of 0.5 to 500 μm, a pore volume in the range of 0.5 to 10 mL/g and a BET surface in the range of 200 to 2000 m²/g.

The invention relates to particulate carbon material provided with elemental silver and elemental ruthenium, and to an efficient process for the preparation thereof.

WO 2021/084140 A2 discloses a process for preparing a particulate carrier material provided with elemental silver and elemental ruthenium, which can be used as an additive for the antimicrobial treatment of a wide variety of materials and substances.

The object of the invention was to provide a highly antimicrobially active material based on a carbon material provided with elemental silver and elemental ruthenium.

The object can be achieved by providing a particulate carbon material that is provided with elemental silver and elemental ruthenium and has an average particle size (d50) in the range of 0.5 to 500 μm, a pore volume in the range of 0.5 to 10 mL/g and a BET surface in the range of 200 to 2,000 m²/g (hereinafter also referred to as “particulate carbon material provided with elemental silver and elemental ruthenium” for short). Its silver-plus-ruthenium weight proportion formed by the elemental silver and the elemental ruthenium can, for example, be in the range of 0.1 to 50 wt. % (% by weight) at a silver: ruthenium weight ratio in the range of 1 to 2,000 parts by weight of silver: 1 part by weight of ruthenium, for example.

The particulate carbon material according to the invention provided with elemental silver and elemental ruthenium substantially consists of carbon particles provided with elemental silver and elemental ruthenium, i.e., it comprises carbon particles provided with elemental silver and elemental ruthenium in a proportion, for example, in the range of 95 to 100 wt. %, in particular 100 wt. %. The possible proportion, not exceeding 5 wt. %, can be formed by carbon particles free of noble metal. In other words, the particulate carbon material according to the invention provided with elemental silver and elemental ruthenium can in particular consist of 95 to 100 wt. % of carbon particles provided with elemental silver and elemental ruthenium and 0 to 5 wt. % of carbon particles free of noble metal, wherein the wt. % add up to 100 wt. %.

The terms “particulate carbon material” and “carbon particle” are used herein. The particulate carbon material or the carbon particles are not carbon nanotubes or carbon nanofibers. Rather, they can be carbon particles, in particular activated carbon particles, i.e., the carbon particles provided with elemental silver and elemental ruthenium can in particular be carbon particles provided with elemental silver and elemental ruthenium, in particular activated carbon particles provided with elemental silver and elemental ruthenium, and the carbon particles free of noble metal can be carbon particles free of noble metal, in particular activated carbon particles free of noble metal.

The term “average particle size” used herein means the volume-average primary particle diameter (d50) determinable by means of laser diffraction. In this case, what is known as Equivalent Circular Area Diameter (ECAD) can advantageously be used as a measure of the particle diameter (cf. RENLIANG XU ET AL: “Comparison of sizing small particles using different technologies,” POWDER TECHNOLOGY, ELSEVIER, BASEL (CH), vol. 132, no. 2-3, Jun. 24, 2003 (Jun. 24, 2003), pages 145-153). Laser diffraction measurements can be carried out using a corresponding particle size measuring instrument, for example a Mastersizer 3000 or Mastersizer 2000 from Malvern Instruments according to the wet determination process. In the wet determination process, a particulate sample can be dispersed in ethanol by means of ultrasound as part of the preparation of the sample.

The term “pore volume” is used herein. The pore volume can be determined by means of mercury porosimetry according to DIN ISO 15901-1:2016 (sample mass 30 mg; surface tension of mercury 0.48 N/m; contact angle of mercury 141.3°; instrument: Porotec Pascal 140+440; measurement process: scanning; start filling pressure 0.0128 MPa; dilatometer: powder, small volume; sample preparation: 8 h at 110° C. under vacuum).

The term “BET surface” used herein refers to the specific surface area that can be determined by means of BET measurement according to DIN ISO 9277:2014-01 (according to chapter 6.3.1, static-volumetric measurement process, gas used: nitrogen).

The invention also relates to a process for preparing such a particulate carbon material provided with elemental silver and elemental ruthenium. From another perspective, the process can also be understood as a process for providing a particulate carbon material with elemental silver and elemental ruthenium. In the process according to the invention, the particulate carbon material according to the invention provided with elemental silver and elemental ruthenium can be obtained by reducing at least one silver precursor and at least one ruthenium precursor in the presence of aqueously suspended carbon particles, in particular activated carbon particles, then separating the solid formed in the process from the aqueous phase, optionally washing the separated solid with water and optionally drying the solid. The process according to the invention accordingly comprises the steps of: reducing at least one silver precursor and at least one ruthenium precursor in the presence of aqueously suspended carbon particles, in particular activated carbon particles; separating the solid formed in the course of the reduction process from the aqueous phase; optionally washing the separated solid with water; and optionally drying the separated and optionally washed solid. During the reduction process, silver and ruthenium precursors can be reduced sequentially or preferably simultaneously.

The silver precursors and ruthenium precursors mentioned herein are silver and ruthenium compounds. Elemental silver or elemental ruthenium can be produced from the silver and ruthenium compounds, respectively, by reduction.

Examples of suitable silver compounds include silver acetate, silver sulfate, and preferably silver nitrate.

Examples of suitable ruthenium compounds include ruthenium oxalate, ruthenium acetate, and in particular ruthenium nitrosyl nitrate.

Particular preference is given to using a combination of silver nitrate and ruthenium nitrosyl nitrate as a combination of precursor compounds in the process according to the invention.

The activated carbon particles used during said reduction process are, in particular, activated carbon particles having an average particle size (d50) in the range of 0.5 to 400 μm, a pore volume in the range of 0.5 to 10 mL/g and a BET surface in the range of 300 to 3,000 m²/g. The activated carbon particles can be of various types, for example pyrolytically produced activated carbon, or in particular those from natural sources such as wood, peat, fruit peels, walnut shells, apricot shells, date skins, coconut shells, and the like. They can be activated carbons comprising a combustion behavior characterized by a weight loss of 50% after 25 to ≤28 minutes, wherein the weight loss is determined by TGA (thermogravimetric analysis) of a small (approximately 8 to 16 mg) powder sample of the corresponding activated carbon in an air atmosphere at a starting temperature of 200° C. and a heating rate of 20° C. per minute. The 100% starting weight of about 8 to 16 mg is determined at the starting temperature of 200° C. Activated carbons made from wood or peat are examples of activated carbons having such combustion behavior. Examples include commercially available types of activated carbon such as Norit® SX Plus, manufactured by Cabot Corporation, Alpharetta, Georgia, and Acticarbone® 3S and Acticarbone® CXV, manufactured by Calgon Carbon Corporation, Moon Township, Pennsylvania. Other activated carbons have poorer combustion behavior that can be characterized by a weight loss of 50% after >28 minutes, for example >28 to 35 minutes, wherein the weight loss is determined by the same TGA measurement process and under the same conditions as mentioned above. If the weight loss of 50% lasts longer than 35 minutes, the temperature is maintained at 800° C. and is not increased above this level. Activated carbon from coconut shells is an example of a type of activated carbon having poor combustion behavior. Examples include commercially available activated carbon types such as Desorex® C33 spezial and Carbopal® CCP 90 FF spezial, both manufactured by Donau Carbon GmbH, Frankfurt, Germany.

The reduction taking place in the process according to the invention can take place either at an acidic pH in the range of 1 to 4, preferably 1 to 2, and at a temperature in the range of 70 to 100° C., preferably 70 to 90° C., with formic acid, or at an acidic pH in the range of 1 to 4, preferably 1 to 2, and at a temperature in the range of 10 to 40° C., preferably 15 to 35° C., with hydrazine, or at a basic pH in the range of 9 to 14, preferably 9 to 12, and at a temperature in the range of 10 to 40° C., preferably 15 to 35° C. with a reducing agent selected from the group consisting of sodium borohydride, hydrazine, hypophosphites and formates.

Hydrazine can be used as such, but preferably as hydrazine hydrate having a hydrazine content in the range of 30 to 65 wt. %. When working at basic pH, hydrazine can also be used as a hydrazinium salt, for example as hydrazinium sulfate. Preference is given to working with hydrazine hydrate.

Hypophosphites and formates are mentioned herein. These are salts, in particular alkali metal salts, alkaline earth salts, and ammonium salts (NH₄ salts). Preference is given to sodium hypophosphite and potassium hypophosphite or sodium formate and potassium formate.

In the process according to the invention, the reducing agent(s) is/are used in a stoichiometrically required amount or more, but preferably in no more than 110% (hydrazine as the reducing agent) or in no more than 200% (formic acid, sodium borohydride, hypophosphites or formates as the reducing agent) of the stoichiometrically required amount in order to completely reduce the silver and ruthenium precursors to elemental silver and elemental ruthenium. Explained using the example of hydrazine, this means that 1 mol of the reducing agent hydrazine can provide 4 mol of electrons having a reducing effect and accordingly releases 1 mol of N₂ in a reduction; accordingly, for example, the reduction of 1 mol of Ag⁺ requires 0.25 mol of hydrazine and the reduction of 1 mol of Ru³⁺ requires 0.75 mol of hydrazine.

Four embodiments of the process according to the invention for preparing the particulate carbon material according to the invention provided with elemental silver and elemental ruthenium are disclosed below.

In a first and preferred embodiment, the preparation process according to the invention comprises the sequential steps of:
(1a) providing an aqueous suspension comprising water, carbon particles, in particular activated carbon particles, at least one silver precursor and at least one ruthenium precursor,
(2a) bringing the aqueous suspension into contact with formic acid at a pH in the range of 1 to 4 and at a temperature in the range of 70 to 100° C., or with hydrazine at a pH in the range of 1 to 4 and at a temperature in the range of 10 to 40° C.,
(3) separating the solid that is formed from the aqueous phase,
(4) optionally washing the separated solid with water, and
(5) optionally drying the solid.

The sequence of steps (1a) to (5) relates to successive steps, and they can be directly successive steps without intermediate steps.

In step (1a) of the first embodiment of the process according to the invention, an aqueous suspension comprising water, carbon particles, in particular activated carbon particles, at least one silver precursor and at least one ruthenium precursor is provided.

The aqueous suspension can be prepared by adding carbon particles, in particular activated carbon particles, to an aqueous solution of the at least one silver precursor and of the at least one ruthenium precursor and suspending them therein.

However, it is preferred to work in such a way that silver precursors and ruthenium precursors, preferably in each case as an aqueous solution, are added simultaneously or in any order (overlapping with a time delay, alternating or successively) to an initially charged aqueous suspension of carbon particles, in particular activated carbon particles. It is particularly preferred that an aqueous solution of both precursors (of the at least one silver precursor and of the at least one ruthenium precursor) is added to an initially charged aqueous suspension of carbon particles, in particular activated carbon particles. The addition preferably takes place slowly, for example over a period of 2 or more hours, for example 3 to 5 hours.

In general, mixing is carried out during and also after the addition, for example by means of stirring. Mixing after the addition can expediently be carried out over a period of, for example, 8 to 120 hours, in particular 24 to 120 hours.

The addition and mixing processes can take place at an elevated temperature, for example in the range of 60 to 90° C. It may also be expedient to carry out mixing at a pH of the aqueous suspension that is in the pH range prevailing in the subsequent step (2a); a suitable agent for correspondingly adjusting the pH can be added for this purpose.

The weight proportion of the carbon particles, in particular activated carbon particles, of the aqueous suspension provided in step (1a) of the first embodiment of the process according to the invention can be, for example, in the range of 5 to 30 wt. %.

The silver weight proportion of the aqueous suspension provided in step (1a) of the first embodiment of the process according to the invention can be in the range of, for example, 0.01 to 2.5 wt. %, while the ruthenium weight proportion can be in the range of, for example, 0.01 to 0.13 wt. %. The aqueous suspension provided in step (1a) of the first embodiment of the process according to the invention is characterized by a weight ratio for example in the range of 1 to 2,000 parts by weight of silver: 1 part by weight of ruthenium and generally significantly in favor of the silver. This silver: ruthenium weight ratio is also found in the product obtained after completion of step (5).

In addition to carbon particles, in particular activated carbon particles and the noble metal precursors, the aqueous suspension provided in step (1a) of the first embodiment of the process according to the invention generally only comprises water and optionally an agent used for adjusting the pH.

In step (2a) of the first embodiment of the process according to the invention, the aqueous suspension provided in step (1a) is brought into contact with formic acid at a pH in the range of 1 to 4, preferably 1 to 2, and at a temperature in the range of 70 to 100° C., preferably 70 to 90° C., or with hydrazine at a pH in the range of 1 to 4, preferably 1 to 2, and at a temperature in the range of 10 to 40° C., preferably 15 to 35° C. If necessary, a corresponding pH and a corresponding temperature of the aqueous suspension can first be adjusted in this case. The acidic pH can be adjusted using acetic acid, for example. The formic acid or hydrazine is used, as mentioned above, in a stoichiometrically required amount or more for the complete reduction of the silver and ruthenium precursors to elemental silver and elemental ruthenium, respectively. Preference is given to working in such a way that the formic acid is added to the aqueous suspension in concentrated form or in the form of, for example, an 80 to <100 wt. % aqueous solution, or the hydrazine is added to the aqueous suspension as such or aqueously diluted with a hydrazine concentration in the range of, for example, 65 to <100 wt. %. The addition preferably takes place slowly, for example over a period of 2 or more hours, for example 3 to 5 hours. In general, mixing is carried out during and also after the addition process, for example by means of stirring, for example over a period of 2 to 24 hours, in particular 4 to 8 hours.

In a second and particularly preferred embodiment, the preparation process according to the invention comprises the sequential steps of:
(1b) providing an aqueous suspension comprising water, carbon particles, in particular activated carbon particles, at least one silver precursor and at least one ruthenium precursor,
(2b) bringing the aqueous suspension into contact with a reducing agent selected from the group consisting of sodium borohydride, hydrazine, hypophosphites and formates at a pH in the range of 9 to 14 and at a temperature in the range of 10 to 40° C.,
(3) separating the solid that is formed from the aqueous phase,
(4) optionally washing the separated solid with water, and
(5) optionally drying the solid.

The sequence of steps (1b) to (5) relates to successive steps, and they can be directly successive steps without intermediate steps.

Step (1b) of the second embodiment of the process according to the invention does not differ from step (1a) of the first embodiment of the process according to the invention or is carried out analogously to step (1a); in this respect, reference is made to the above statements regarding step (1a) but by only pointing out that it can also be expedient here to carry out mixing at a pH of the aqueous suspension that is in the pH range prevailing in the subsequent step (here (2b)); a suitable agent for correspondingly adjusting the pH can be added for this purpose.

It is particularly expedient in step (1b) to work with an activated carbon having an inherently alkaline pH, for example Ceca 2SW activated carbon from Chemviron.

In step (2b) of the second embodiment of the process according to the invention, the aqueous suspension provided in step (1b) is brought into contact with a reducing agent selected from the group consisting of sodium borohydride, hydrazine, hypophosphites and formates at a pH in the range of 9 to 14, preferably 9 to 12, and at a temperature in the range of 10 to 40° C., preferably 15 to 35° C. If necessary, a corresponding pH and a corresponding temperature of the aqueous suspension can first be adjusted in this case. The alkaline pH can be adjusted using a strong base, in particular alkali hydroxide, especially sodium hydroxide or potassium hydroxide. The reducing agent is used, as mentioned above, in a stoichiometrically required amount or more for the complete reduction of the silver and ruthenium precursors to elemental silver and elemental ruthenium, respectively. Preference is given to working in such a way that the reducing agent is added to the aqueous suspension in the form of an aqueous solution. The addition preferably takes place slowly, for example over a period of 2 or more hours, for example 3 to 5 hours. In general, mixing is carried out during and also after the addition, for example by means of stirring, for example over a period of 2 to 24 hours, in particular 4 to 8 hours.

In a third embodiment, the preparation process according to the invention comprises the sequential steps of:
(1c) providing an aqueous suspension having a pH in the range of 9 to 14 comprising water, carbon particles, in particular activated carbon particles, and a reducing agent selected from the group consisting of sodium borohydride, hydrazine, hypophosphites and formates,
(2c) bringing the aqueous suspension into contact (i) with an aqueous solution comprising at least one silver precursor and at least one ruthenium precursor or (ii) with an aqueous solution comprising at least one silver precursor and then with an aqueous solution comprising at least one ruthenium precursor or (iii) with an aqueous solution comprising at least one ruthenium precursor and then with an aqueous solution comprising at least one silver precursor at a pH in the range of 9 to 14 and at a temperature in the range of 10 to 40° C.,
(3) separating the solid that is formed from the aqueous phase,
(4) optionally washing the separated solid with water, and
(5) optionally drying the solid.

The sequence of steps (1c) to (5) relates to successive steps, and they can be directly successive steps without intermediate steps.

In step (1c) of the third embodiment of the process according to the invention, an aqueous suspension having a pH in the range of 9 to 14, preferably 9 to 12, comprising water, carbon particles, in particular activated carbon particles, and a reducing agent selected from the group consisting of sodium borohydride, hydrazine, hypophosphites and formates is provided. The aqueous suspension can be prepared by adding carbon particles, in particular activated carbon particles, to an aqueous solution of the reducing agent and suspending them therein. However, preference is given to working in such a way that the reducing agent is added, preferably as an aqueous solution, to an initially charged aqueous suspension of carbon particles, in particular activated carbon particles. The alkaline pH can be adjusted using a strong base, in particular alkali hydroxide, especially sodium hydroxide or potassium hydroxide. In general, mixing is carried out during and also after the addition, for example by means of stirring, for example over a period of 1 to 2 hours.

The weight proportion of the carbon particles, in particular activated carbon particles, of the aqueous suspension provided in step (1c) of the third embodiment of the process according to the invention can be, for example, in the range of 5 to 30 wt. %.

The weight proportion of the reducing agent of the aqueous suspension provided in step (1c) of the third embodiment of the process according to the invention can be in the range of, for example, 0.5 to 10 wt. %.

In addition to carbon particles, in particular activated carbon particles and the reducing agent, the aqueous suspension provided in step (1c) of the third embodiment of the process according to the invention generally only comprises water and optionally a base used for adjusting the pH.

It is particularly expedient in step (1c) to work with an activated carbon having an inherently alkaline pH, for example Ceca 2SW activated carbon from Chemviron.

In step (2c) of the third embodiment of the process according to the invention, the aqueous suspension provided in step (1c) is brought into contact (i) with an aqueous solution comprising at least one silver precursor and at least one ruthenium precursor or (ii) with an aqueous solution comprising at least one silver precursor and then with an aqueous solution comprising at least one ruthenium precursor or (iii) with an aqueous solution comprising at least one ruthenium precursor and then with an aqueous solution comprising at least one ruthenium precursor at a pH in the range of 9 to 14, preferably 9 to 12, and at a temperature in the range of 10 to 40° C., preferably 15 to 35° C. The single reducing agent is used, as mentioned above, in a stoichiometrically required amount or more for the complete reduction of the silver and ruthenium precursors to elemental silver and elemental ruthenium, respectively. Preference is given to working according to variant (i).

The noble metal weight proportion formed of silver and/or ruthenium of the aqueous noble metal precursor solutions used according to variants (i), (ii) or (iii) in step (1c) of the third embodiment of the process according to the invention can be in the range of, for example, 1 to 40 wt. %. A weight ratio in the range of, for example, 1 to 2,000 parts by weight of silver: 1 part by weight of ruthenium is used and is in this case generally significantly in favor of the silver. This silver: ruthenium weight ratio is also found in the product obtained after completion of step (5).

In general, mixing is carried out during and also after the addition, for example by means of stirring, for example over a period of 2 to 24 hours, in particular 4 to 8 hours.

In a fourth embodiment, the preparation process according to the invention comprises the sequential steps of:
(1d) providing an aqueous suspension having a pH in the range of 1 to 4 comprising water, carbon particles, in particular activated carbon particles and formic acid or hydrazine,
(2d) bringing the aqueous suspension into contact (i) with an aqueous solution comprising at least one silver precursor and at least one ruthenium precursor or (ii) with an aqueous solution comprising at least one silver precursor and then with an aqueous solution comprising at least one ruthenium precursor or (iii) with an aqueous solution comprising at least one ruthenium precursor and then with an aqueous solution comprising at least one silver precursor at a pH in the range of 1 to 4 and at a temperature in the range of 70 to 100° C. (in the case of formic acid as a reducing agent) or at a temperature in the range of 10 to 40° C. (in the case of hydrazine as a reducing agent),
(3) separating the solid that is formed from the aqueous phase,
(4) optionally washing the separated solid with water, and
(5) optionally drying the solid.

The sequence of steps (1d) to (5) relates to successive steps, and they can be directly successive steps without intermediate steps.

In step (1d) of the fourth embodiment of the process according to the invention, an aqueous suspension having a pH in the range of 1 to 4, preferably 1 to 2, comprising water, carbon particles, in particular activated carbon particles, and formic acid or hydrazine is provided.

The aqueous suspension can be prepared by adding carbon particles, in particular activated carbon particles, to an aqueous solution of the formic acid or hydrazine and suspending them therein. However, preference is given to working in such a way that the formic acid or the hydrazine is added, preferably as an aqueous solution, to an initially charged aqueous suspension of carbon particles, in particular activated carbon particles. The acidic pH can be adjusted using acetic acid, for example. In general, mixing is carried out during and also after the addition, for example by means of stirring, for example over a period of 1 to 2 hours.

The weight proportion of the carbon particles, in particular activated carbon particles, of the aqueous suspension provided in step (1d) of the fourth embodiment of the process according to the invention can be, for example, in the range of 5 to 30 wt. %.

The weight proportion of the formic acid or hydrazine of the aqueous suspension provided in step (1d) of the fourth embodiment of the process according to the invention can be in the range of, for example, 0.5 to 10 wt. %.

In addition to carbon particles, in particular activated carbon particles, and formic acid or hydrazine, the aqueous suspension provided in step (1d) of the fourth embodiment of the process according to the invention generally only comprises water and optionally an acid used for adjusting the pH.

In step (2d) of the fourth embodiment of the process according to the invention, the aqueous suspension provided in step (1d) is brought into contact (i) with an aqueous solution comprising at least one silver precursor and at least one ruthenium precursor or (ii) with an aqueous solution comprising at least one silver precursor and then with an aqueous solution comprising at least one ruthenium precursor or (iii) with an aqueous solution comprising at least one ruthenium precursor and then with an aqueous solution comprising at least one silver precursor at a pH in the range of 1 to 4, preferably 1 to 2, and at a temperature in the range of 70 to 100° C., preferably 70 to 90° C. in the case of formic acid as a reducing agent, or at a pH in the range of 1 to 4, preferably 1 to 2, and at a temperature in the range of 10 to 40° C., preferably 15 to 35° C., in the case of hydrazine as reducing agent. In this case, the formic acid or hydrazine is used, as mentioned above, in a stoichiometrically required amount or more for the complete reduction of the silver and ruthenium precursors to elemental silver and elemental ruthenium, respectively. Preference is given to working according to variant (i).

The noble metal weight proportion formed from silver and/or ruthenium of the aqueous noble metal precursor solutions used according to variants (i), (ii) or (iii) in step (1d) of the fourth embodiment of the process according to the invention can be in the range of, for example, 1 to 36 wt. %. A weight ratio in the range of, for example, 1 to 2,000 parts by weight of silver: 1 part by weight of ruthenium is used and is in this case generally significantly in favor of the silver. This silver: ruthenium weight ratio is also found in the product obtained after completion of step (5).

In general, mixing is carried out during and also after the addition, for example by means of stirring, for example over a period of 2 to 24 hours, in particular 4 to 8 hours.

Steps (3) to (5) are the same in all four embodiments of the process according to the invention.

In step (3), the solid formed in each of steps (2a), (2b), (2c) and (2d) is separated from the aqueous phase. Examples of solid-liquid separation processes suitable for this purpose include processes known to a person skilled in the art such as decanting, pressing, filtering, suction filtration, centrifugation, or combinations thereof.

Step (4) of the process according to the invention is an optional, but generally expedient step in which the solid separated in step (3) can be washed with water. Water-soluble constituents can be removed here. Washing can take place, for example, on a Nutsche filter.

Step (5) of the process according to the invention is an optional, but generally expedient step in which the solid separated in step (3) and optionally washed in step (4) can be dried. In this case, water and any other volatile constituents present are removed from the solid obtained after completion of step (3) or step (4). The water can be removed in the sense of virtually complete water removal or in the sense of water removal until a desired residual moisture content is reached. For this purpose, most of the water can be removed by conventional processes, such as pressing, press filtration, suction filtration, centrifugation or similar processes, before drying, optionally supported by reduced pressure, is carried out at temperatures in the range of, for example, 20 to 150° C.

After completion of step (5), the particulate carbon material according to the invention that is provided with elemental silver and elemental ruthenium and has an average particle size (d50) in the range of 0.5 to 500 μm, a pore volume in the range of 0.5 to 10 mL/g and a BET surface in the range of 200 to 2,000 m2/g is obtained. Its silver-plus-ruthenium weight proportion formed by the elemental silver and the elemental ruthenium can vary within wide limits, for example in the range of 0.1 to 50 wt. %, preferably 1 to 40 wt. %, and at the same time the silver: ruthenium weight ratio can be in the range of, for example, 1 to 2,000 parts by weight of silver: 1 part by weight of ruthenium. Scanning electron micrographs can show that the silver and the ruthenium are present on inner surfaces (within pores and/or cavities) and/or on the outer surface of the originally silver-free and ruthenium-free carbon particles, and can thereby form, for example, a discontinuous layer and/or small silver or ruthenium particles (silver or ruthenium islands). The silver and the ruthenium are not alloyed, but are randomly distributed, and both noble metals are at least partially in contact with one another. It is clear to a person skilled in the art that the silver and the ruthenium can comprise other silver species than elemental metal silver and other ruthenium species than elemental metal ruthenium on the surface thereof—for example corresponding oxides, halogenides and/or sulfides. Such species can be formed during the performance of the process according to the invention or thereafter, for example during storage, use or further processing of the particulate carbon material according to the invention provided with elemental silver and elemental ruthenium.

The particulate carbon material according to the invention provided with elemental silver and elemental ruthenium is characterized by a dark or black color with a correspondingly low brightness L*, for example in the range of 35 to 45, which can be disturbing for some applications. The brightness L* is a specific* L* in the CIEL*a*b* color space (DIN EN ISO/CIE 11664-4:2020-03) determined by spectrophotometry at a measurement geometry of d/8°; the spectrophotometric measurement of the particulate carbon material according to the invention provided with elemental silver and elemental ruthenium can be performed on a sample poured into a colorless glass vessel to a filling height of 1 cm through the flat glass bottom of the glass vessel placed on the measuring head of the spectral photometer used.

If desired, the particulate carbon material according to the invention provided with elemental silver and elemental ruthenium can be further processed to form a brightened particulate material having a brightness L*, for example in the range of 50 to 85. For brightening purposes, the particulate carbon material according to the invention provided with elemental silver and elemental ruthenium can be brought into contact with at least one C1-C4 alkoxide of aluminum, magnesium, calcium, silicon, zinc, zirconium, and/or titanium in the presence of an amount of water that is at least sufficient for complete hydrolysis of the at least one C1-C4 alkoxide. As stated, a brightened particulate material, i.e., a particulate material having a color, for example a gray color, with a brightness L* in the range of, for example, 50 to 85 can be formed. This brightened particulate material consists of the particulate carbon material according to the invention provided with elemental silver and elemental ruthenium having a solid located at least partially thereon. Depending on the selection of the at least one C1-C4 alkoxide, the solid is a solid selected from the group consisting of aluminum oxide, aluminum hydroxide, aluminum oxyhydroxide, magnesium oxide, magnesium hydroxide, magnesium oxyhydroxide, calcium oxide, calcium hydroxide, calcium oxyhydroxide, silicon dioxide, silica, zinc oxide, zinc hydroxide, zinc oxyhydroxide, zirconium dioxide, zirconium(IV) oxyhydrates, titanium dioxide, titanium(IV) oxyhydrates, and combinations thereof.

The particulate carbon material according to the invention provided with elemental silver and elemental ruthenium, optionally brightened as mentioned above, is characterized by a particularly high antimicrobial effect, as can be determined in conventional inhibition zone tests or by determining the minimum inhibitory concentration from growth curves of microorganisms. In this respect, the invention also relates to the use of the optionally brightened particulate carbon material according to the invention provided with elemental silver and elemental ruthenium as an additive for the antimicrobial treatment of metal surfaces; coating agents; plasters; molding compounds; plastics materials in the form of plastics films, plastics parts, or plastics fibers; synthetic resin products; ion-exchange resins; silicone products; cellulose-based products; foams; cosmetics; and many others.

The use of the optionally brightened particulate carbon material according to the invention provided with elemental silver and elemental ruthenium as an additive is particularly expedient for the antimicrobial treatment of filter fleeces; of textiles or in textile applications; in health and hygiene applications, for example in face masks, breathing masks, and insoles; of products provided with or based on activated carbon, such as a plastics material provided with activated carbon as an additive; of activated carbon filters usable for air or water purification; or of products comprising activated carbon filters.

The particulate carbon material according to the invention provided with elemental silver and elemental ruthenium, optionally brightened as mentioned above, can also be used as a heterogeneous catalyst, for example in the catalysis of the formation of hydroxyl radicals in aqueous media permitting bacterial growth.

### EXAMPLES

### Reference Example 1 (Preparation of a Cellulose Powder Provided with 18.9 wt. % of Elemental Silver and 1.0 wt. % of Elemental Ruthenium)

75.6 g (445 mmol) of solid silver nitrate and 13.94 g of ruthenium nitrosyl nitrate solution (ruthenium content 19.0 wt. %; 26.2 mmol Ru) were dissolved in 416.8 g of deionized water, and the aqueous precursor solution obtained in this way was mixed homogeneously with 211.2 g of cellulose powder (Vitacel® L-600 from Rettenmaier und Söhne GmbH & Co KG) to form an orange, free-flowing impregnated particulate material. 705 mL of an aqueous hydrazine solution [4.19 g (131 mmol) of hydrazine and 81.81 g of a 32 wt. % sodium hydroxide solution (654.51 mmol NaOH), remainder: water] having a pH of 13.9 were metered into the free-flowing impregnated particulate material at room temperature and a metering rate of 30 mL/min while stirring. Over time, a homogeneous pulp that became easier to stir was formed. After the metering had ended, stirring was continued for 30 minutes until nitrogen release could no longer be observed. The material was then filtered off by means of suction, washed with a total of 1,000 mL of water, and dried in a drying cabinet at 105° C./300 mbar to a residual moisture content of 15 wt. %. A silver content of 18.9 wt. % and a ruthenium content of 1.0 wt. % of the end product (relative to 0 wt. % residual moisture) was determined by means of ICP-OES.

### Example 2 According to the Invention (Preparation of a Particulate Carbon Material Provided with 18.7 wt. % of Elemental Silver and 0.9 wt. % of Elemental Ruthenium)

An aqueous solution prepared from 157.2 g of aqueous silver nitrate solution (silver content 36.2 wt. %; 528 mmol Ag) and 14.9 g of aqueous ruthenium nitrosyl nitrate solution (ruthenium content 18.7 wt. %; 30 mmol Ru) and 1,000 mL of water was added over a period of 3 hours (7 mL/min) to a suspension of 240 g of activated carbon (Ceca 2SW) in 3,000 mL of deionized water. The pH of the suspension was adjusted to 9 using 32 wt. % aqueous sodium hydroxide solution. This suspension was stirred at 80° C. for 2 days. The suspension was then cooled to 35° C., a solution consisting of 7.7 g of hydrazine hydrate (hydrazine content 64 wt. %), 96.5 g of sodium hydroxide and 400 mL of water was metered in over a period of 3 hours (3 mL/min) and stirred for a further 4 hours. The material was then filtered off with suction and washed with a total of 20 L of water. In the product obtained in this way, a residual water content of 50.8 wt. % was determined. A silver content of 18.7 wt. % and a ruthenium content of 0.9 wt. % of the product (relative to 0 wt. % residual moisture) was determined by means of ICP-OES.

### Example 3 (Test of the Products From Reference Example 1 and Example 2 According to the Invention to Compare Their Antimicrobial Effect)

In separate Erlenmeyer flasks, 30 mL of a culture of methicillin-resistant Staphylococcus aureus (MRSA) in trypic soy broth (TSB) was adjusted to an optical density of 0.05. Different amounts of the product from Reference Example 1 in the range of 1 to 20 mg were then weighed in. The samples were incubated in a shaking incubator at 37° C. and 150 rpm. Within 6 hours, the optical density at a wavelength of 600 nm (OD600) was determined at hourly intervals. The inhibition of bacterial growth was indicated by a reduced increase in optical density compared to the control sample. An MRSA culture without the addition of an active antimicrobial substance served as the control sample. In the case of complete inhibition of bacterial growth, no increase in optical density was to be observed. The corresponding sample amount of the product from Reference Example 1 or Example 2 according to the invention was used to calculate the minimum inhibitory concentration. This resulted in a minimum inhibitory concentration for the product from Reference Example 1 of 0.55 mg/mL and a lower minimum inhibitory concentration for the product from Example 2 according to the invention of 0.35 mg/mL compared thereto.

1. A particulate carbon material equipped with elemental silver and elemental ruthenium with an average particle size (d50) in the range of 0.5 to 500 pm, a pore volume in the range of 0.5 to 10 mL/g and a BET surface area in the range of 200 to 2000 m²/g.

2. The material according to claim 1 having a silver plus ruthenium weight fraction formed by the elemental silver and the elemental ruthenium in the range of 0.1 to 50 wt. % with a silver: ruthenium weight ratio in the range of 1 to 2000 parts by weight silver: 1 part by weight ruthenium.

3. The material according to claim 1, consisting of 95 to 100 wt. % of carbon particles equipped with elemental silver and elemental ruthenium and 0 to 5 wt. % of noble metal-free carbon particles, the wt. % adding up to 100 wt. %.

4. A process for producing a material according to claim 1, comprising the steps of: reducing at least one silver precursor and at least one ruthenium precursor in the presence of aqueously suspended carbon particles, in particular activated carbon particles, separating the solid formed in the course of the reduction from the aqueous phase, optionally washing the separated solid with water and optionally drying the separated and optionally washed solid.

5. The process according to claim 4, wherein silver and ruthenium precursors are reduced sequentially or simultaneously.

6. The process according to claim 4, wherein the at least one silver precursor is selected from the group consisting of silver acetate, silver sulfate and silver nitrate and wherein the at least one ruthenium precursor is selected from the group consisting of ruthenium oxalate, ruthenium acetate and ruthenium nitrosyl nitrate.

7. The process according to claim 4, wherein the activated carbon particles are activated carbon particles having an average particle size (d50) in the range of 0.5 to 400 pm, a pore volume in the range of 0.5 to 10 mL/g and a BET surface area in the range of 300 to 3000m²/g.

8. The process according to claim 4, wherein the reduction is carried out either at an acidic pH in the range from 1 to 4 and at a temperature in the range from 70 to 100° C. with formic acid, or at an acidic pH in the range from 1 to 4 and at a temperature in the range from 10 to 40° C. with hydrazine, or at a basic pH in the range from 9 to 14 and at a temperature in the range from 10 to 40° C. with a reducing agent selected from the group consisting of sodium borohydride, hydrazine, hypophosphites and formates.

9. The process according to claim 8 in one embodiment comprising the successive steps:
(1a) providing an aqueous suspension comprising water, carbon particles, in particular activated carbon particles, at least one silver precursor and at least one ruthenium precursor,
(2a) contacting the aqueous suspension at a pH in the range from 1 to 4 and at a temperature in the range from 70 to 100° C. with formic acid or at a pH in the range from 1 to 4 and at a temperature in the range from 10 to 40° C. with hydrazine,
(3) separating the solid formed from the aqueous phase,
(4) if necessary, washing the separated solid, and
(5) if necessary, drying the solid.

10. The process according to claim 8 in one embodiment comprising the successive steps:
(1b) providing an aqueous suspension comprising water, carbon particles, in particular activated carbon particles, at least one silver precursor and at least one ruthenium precursor,
(2b) contacting the aqueous suspension at a pH in the range from 9 to 14 and at a temperature in the range from 10 to 40° C. with a reducing agent selected from the group consisting of sodium borohydride, hydrazine, hypophosphites and formates,
(3) sSeparating the solid formed from the aqueous phase,
(4) if necessary, washing the separated solid, and
(5) if necessary, drying the solid.

11. The process according to claim 8 in one embodiment comprising the successive steps:
(1c) providing an aqueous suspension having a pH in the range of 9 to 14 comprising water, carbon particles, in particular activated carbon particles and a reducing agent selected from the group consisting of sodium borohydride, hydrazine, hypophosphites and formates,
(2c) contacting the aqueous suspension at a pH in the range of 9 to 14 and at a temperature in the range of 10 to 40° C. (i) with an aqueous solution comprising at least one silver precursor and at least one ruthenium precursor or (ii) with an aqueous solution comprising at least one silver precursor and then with an aqueous solution comprising at least one ruthenium precursor or (iii) with an aqueous solution comprising at least one ruthenium precursor and then with an aqueous solution comprising at least one silver precursor,
(3) separating the solid formed from the aqueous phase,
(4) if necessary, washing the separated solid, and
(5) if necessary, drying the solid.

12. The process according to claim 8 in one embodiment comprising the successive steps:
(1d) providing an aqueous suspension having a pH in the range of 1 to 4 comprising water, carbon particles, in particular activated carbon particles and formic acid or hydrazine,
(2d) contacting the aqueous suspension at a pH in the range of 1 to 4 and at a temperature in the range of 70 to 100° C. (in the case of formic acid as reducing agent) or at a temperature in the range of 10 to 40° C. (in the case of hydrazine as reducing agent) (i) with an aqueous solution comprising at least one silver precursor and at least one ruthenium precursor or (ii) with an aqueous solution comprising at least one silver precursor and then with an aqueous solution comprising at least one ruthenium precursor or (iii) with an aqueous solution comprising at least one ruthenium precursor and then with an aqueous solution comprising at least one silver precursor,
(3) separating the solid formed from the aqueous phase,
(4) if necessary, washing the separated solid, and
(5) if necessary, drying the solid.

13. The process according to claim 4, wherein the particulate carbon material provided with elemental silver and elemental ruthenium is further processed to a brightened particulate material having a brightness L* in the range of 50 to 85 by treating it with at least one C1-C4 alkoxide of aluminium, Magnesium, calcium, silicon, zinc, zirconium and/or titanium in the presence of an amount of water at least sufficient for complete hydrolysis of the at least one C1-C4 alkoxide.

14. A use of a particulate carbon material provided with elemental silver and elemental ruthenium with an average particle size (d50) in the range of 0.5 to 500 pm, and a pore volume in the range of 0.5 to 10 mL/g and a BET surface area in the range of 200 to 2000 m²/g, or produced by the process of claim 4 as an additive for the antimicrobial treatment of metal surfaces; of coating agents; of plasters; of molding compounds; of plastics in the form of plastic films, plastic parts, plastic fibers or of plastic provided with activated carbon as an additive; synthetic resin products; ion exchange resins; silicone products; cellulose-based products; foams; cosmetics; filter fleeces; textiles; in textile applications; in health and hygiene applications; of face masks; of breathing masks; of insoles; of products provided with or based on activated carbon; of activated carbon filters that can be used for air or water purification; of products comprising activated carbon filters.

15. A use of a particulate carbon material provided with elemental silver and elemental ruthenium with an average particle size (d50) in the range of 0.5 to 500 pm, and a pore volume in the range of 0.5 to 10 mL/g and a BET surface area in the range of 200 to 2000 m²/g, or produced by the process of claim 4, heterogenous catalyst in catalyzing the formation of hydroxyl radicals within aqueous media permitting bacterial growth.
